(19) United States
(12) Patent Application Publication
(10) Pub. No.: US 2006/0025808 A1
(43) Pub. Date: Feb. 2, 2006

(54) Vagal nerve bulking arrangement

(76) Inventors: Ronald J. Thompson, Fort Thomas, KY (US); James M. Thompson, Cincinnati, OH (US)

Correspondence Address: Donald N. Halgren, 35 Central Street, Manchester, MA, 01944, US

(21) Appl. No.: 11/165,075

(22) Filed: Jun. 22, 2005

Related U.S. Application Data
(60) Provisional application No. 60/582,056, filed on Jun. 22, 2004.

(51) Int. Cl. A61B 17/00

(52) U.S. Cl. 606/204

(57) ABSTRACT

A method for the appetite suppression of an individual comprising the steps of introducing a bulking material between the vagal nerve area and the stomach wall of a patient undergoing appetite suppression treatment to reduce production levels of ghrelin in the patient's system.

### BACKGROUND OF THE INVENTION

Field of the Invention

This invention relates to a treatment for reducing the appetite of a patient needing weight loss and more particularly to a medical arrangement related to effecting the vagal nerve to suppress appetite, and is based upon Provisional patent application 60/582,056, filed Jun. 22, 2004, and is incorporated herein by reference in its entirety.

### BRIEF DESCRIPTION OF THE DRAWINGS

The objects and advantages of the invention will become more apparent when viewed in conjunction with the following drawings, in which:

FIG. 1 is a representation of a human stomach in a side elevational view;

FIG. 2 is a view similar to FIG. 1, showing the vagal nerve trunk in the lesser curvature of the stomach;

FIG. 2A is an enlarged representation of the vagal trunk serusa and stomach wall shown in FIG. 2, in a sectional view;

FIG. 3 is a sectional view of the gastroesophageal junction, again showing the vagal nerve trunk;

FIG. 4 is an enlarged sectional view of the stomach wall and vagal nnerve trunk with a gastroscope introducing a bulking agent into a site adjacent the vagal nerve trunk to compress and displace the vagal nerve;

FIG. 5 is an enlarged sectional view of the stomach wall and vagal nerve with a bulking agent disposed therebetween;

FIG. 6 is a sectional view of a distended stomach;

FIG. 6A is an enlarged sectional view of an emplaced bulking agent represented in a portion of the distended stomach shown in FIG. 6;

FIG. 7 is a sectional view of a contracted stomach;

FIG. 7A is an enlarged sectional view of an emplaced bulking agent represented in a portion of the contracted stomach shown in FIG. 6;

FIG. 8 is a sectional view representing an electrical pulse generator implanted in the abdominal wall;

FIG. 8a is an enlarged sectional view of an implanted electrical pacemaker electrode shown in FIG. 8, adjacent the vagal nerve;

FIG. 9a is a sectional view of a preprandial (before meal) stomach;

FIG. 9B is a sectional representation of ghrelin cells and ghrelin in the gastric layer of the stomach represented in FIG. 9A;

FIG. 9C is a sectional view of an enlarged “hungry” stomach which is empty;

FIG. 9D is a sectional representation of empty “spent” ghrelin cells and a ghrelin hormone as it is released into the bloodstream to create hunger and appetite at the hypothalmus in the brain;

FIG. 10A is a sectional representation of the ghrelin cells in the gastric layer of a stomach, depicting 90% of those cells being capable of producing ghrelin (before the vagal nerve trunk bulking)

FIG. 10b is a sectional representation of the ghrelin cells in the gastric layer of a stomach, depicting only 70% of the ghrelin cells capable of producing ghrelin, 30% of the ghrelin cells becoming atrophied one month after vagal nerve bulking, because of decreased drawing power of ghrelin through the vagal trunk; and

FIG. 10C is a sectional representation of the ghrelin cells in the gastric layer of a stomach, depicting only 40% of the ghrelin cells capable of producing ghrelin, with 60% of the ghrelin cells atrophied and inactive after three months from a vagal nerve trunk bulking procedure.

### BRIEF SUMMARY OF THE INVENTION

The present invention relates to a medical procedure wherein the Vagal nerve, in the area of the lesser area of the stomach of a patient needing appetite suppression, has a bio-compatable bulking agent introduced between the trunk of the Vagal nerve and the glandular layer of the stomach wall at the lesser curvature of the stomach.

The bulking material effects a displacement of the Vagal nerve and the bulking material also effects a compression of that Vagal nerve. Such compression and displacement of the Vagal nerve has the effect of suppressing the production of Ghrelin cells which are otherwise introduced into the bloodstream and which otherwise increase an individual's appetite.

Further embodiment of invention is where vagal nerve overide is accomplished by electromechanical pacing, for example a piezoelectric implant to effect the biomaterial, and create electrical energy during stomach contractions. This interferes with the normal electrical impulses of the vagus nerve.

The invention thus comprises a method for the appetite suppression of an individual comprising one or more of the steps of: introducing a bulking material between the vagal nerve area and the stomach wall of a patient undergoing appetite suppression treatment to reduce production levels of ghrelin, wherein the vagal nerve area comprises the vagal trunk and the glandular layer of the stomach. The vagal nerve area may also comprise the vagal nerve arrayed in the lesser curvature of the stomach. The steps may include introducing a gastroscope through the gastroesophageal junction of a patient so as to inject a bulking agent adjacent the vagal nerve trunk to displace and compress the vagal nerve to reduce ghrelin levels in the patient's bloodstream.

The invention may also include a method for the appetite suppression of a patient, comprising displacing the vagal nerve trunk of the patient to effect a reduction in ghrelin levels in the system of the patient.

The invention may also include a method for the appetite suppression of a patient comprising: compressing the vagal nerve trunk of a patient to effect a reduction in ghrelin levels in the system of the patient via piezoelectric stimulation.

### DETAILED DESCRIPTION OF THE PREFERRED EMBODIMENTS

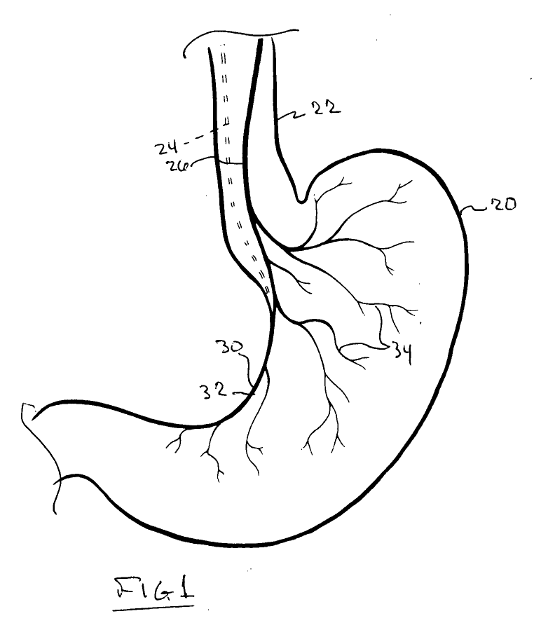

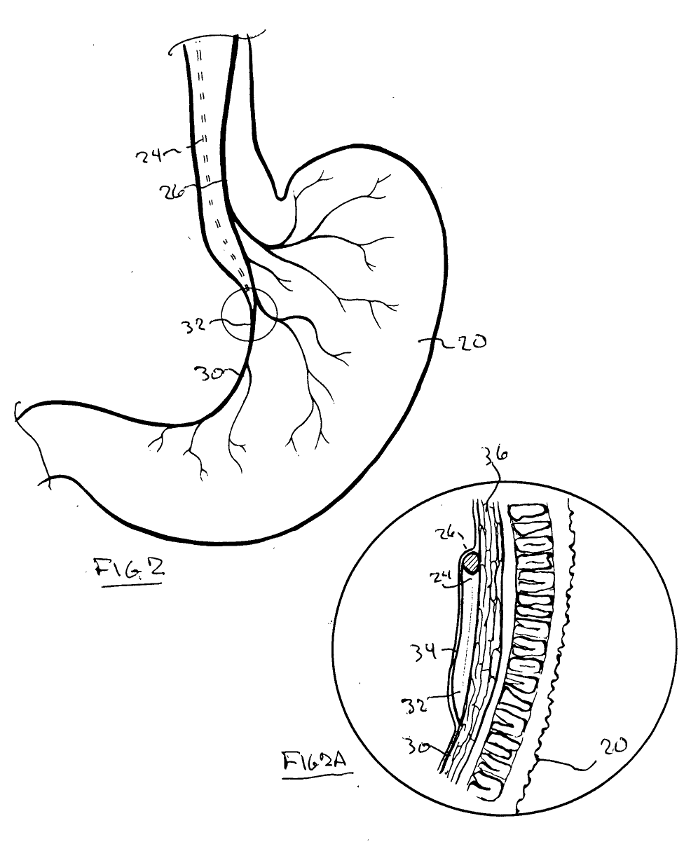

Referring now to the drawings in detail, and particularly to FIG. 1, there is shown a representation of a human stomach 20 in a side elevational view with the esophagus 22 in communication therewith. A right and a left vagus trunk 24 and 26 are represented extending through the esophagus 22 and joining the lesser curvature 30 of the stomach 20. The vagus nerve 32 is shown with nerve branches 34 which innervate the entire stomach 20. In the lesser curvature of the stomach 30, the right and left vagal trunks 24 and 26 course parallel to one another, and are referred to as the “pacemaker” of the stomach. In FIG. 2 shows a view similar to FIG. 1, showing the vagal nerve trunks 24 and 26 joining in the lesser curvature 30 of the stomach 20. FIG. 2A represents an enlarged representation of the vagal trunk serusa and stomach wall shown in FIG. 2, in a sectional view, with the right vagal trunk 24 and serosa 34 on the muscular layer 36 of the wall of the stomach 20.

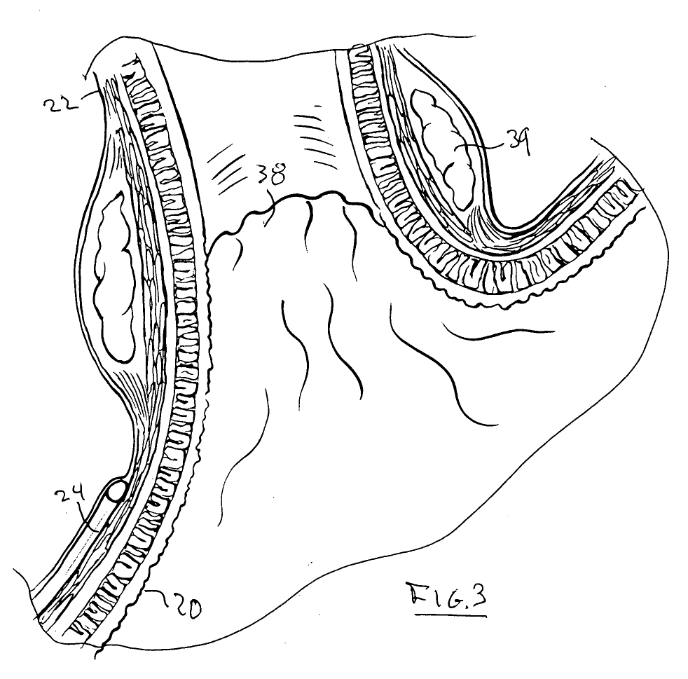

In FIG. 3 is shown a sectional view of the gastroesophageal junction 38, disclosing placement of an enteryx bio material 39 theresurrounding, typically for the treatment of gastro esophageal reflux disease (GERD), and again showing the right vagal nerve trunk 24.

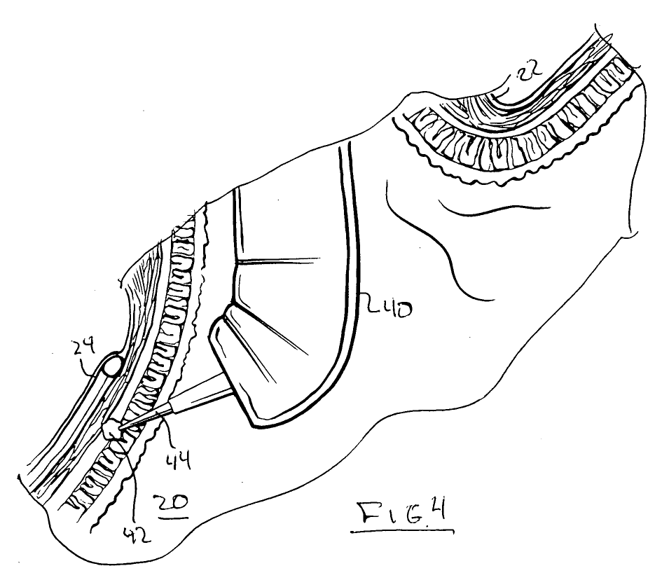

FIG. 4 discloses an enlarged sectional view of the stomach wall and a vagal nerve trunk 24 with a gastroscope 40 through the esophagus 22, introducing a bulking agent 42 through a needle 44 into a site adjacent the vagal nerve trunk 24 and/or 26 to compress and displace the vagas nerve 32 and/or trunks 24 and 26. Placement of a bulking biomaterial 42 compresses the vagal nerve 32 trunks 24 and 26 in the lesser curvature 30 of the stomach 20. The bulking material 42 also displaces the vagal nerve trunks 24 and 26.

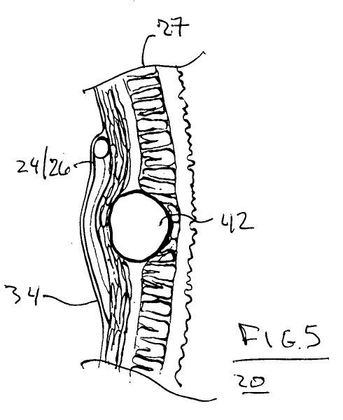

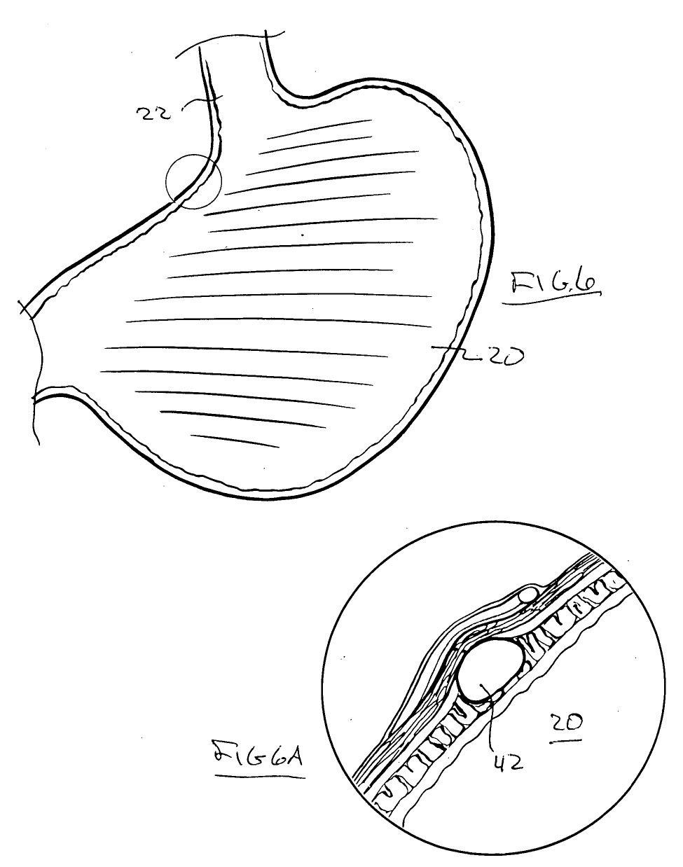

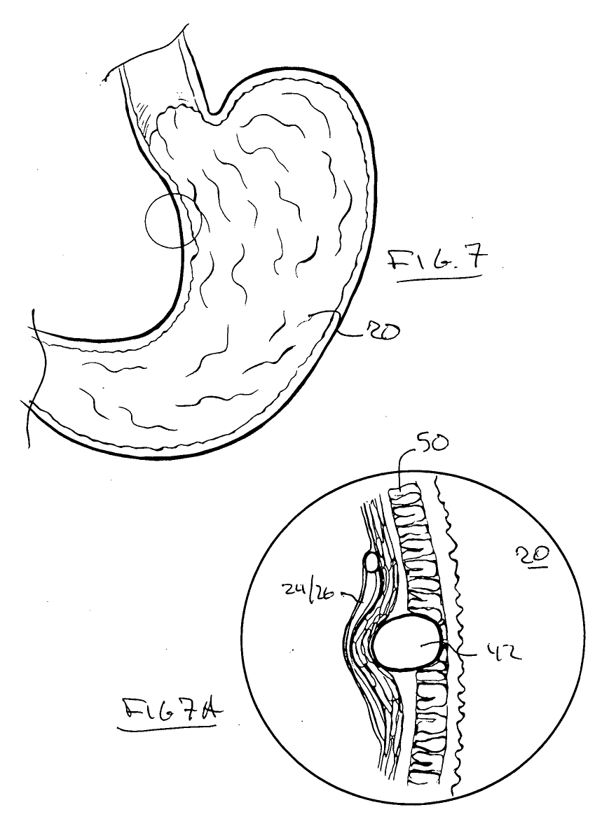

FIG. 5 shows an enlarged sectional view of the stomach wall and vagal nerve 32 with a bulking agent 42 disposed between the vagal nerve 32 or 24/26 and the glandular layer 27, compressing and displacing the vagal nerve trunks 24/26 at the “pacemaker” of the stomach 20. FIG. 6 shows a sectional view of a distended stomach 20. FIG. 6A shows an enlarged sectional view of an emplaced bulking agent 42 represented in a portion of the distended stomach 20 shown in FIG. 6. The view in FIG. 7 is a sectional view of a stomach 20 in a contracted state, with the greater curvature 48 and the lesser curvature 30 shown. FIG. 7A represents an enlarged sectional view of an emplaced bulking agent 42 represented in a portion of the contracted stomach shown in FIG. 6, biasing both the vagal nerve trunk 24/26 and the mucosa 50 during a stomach contraction.

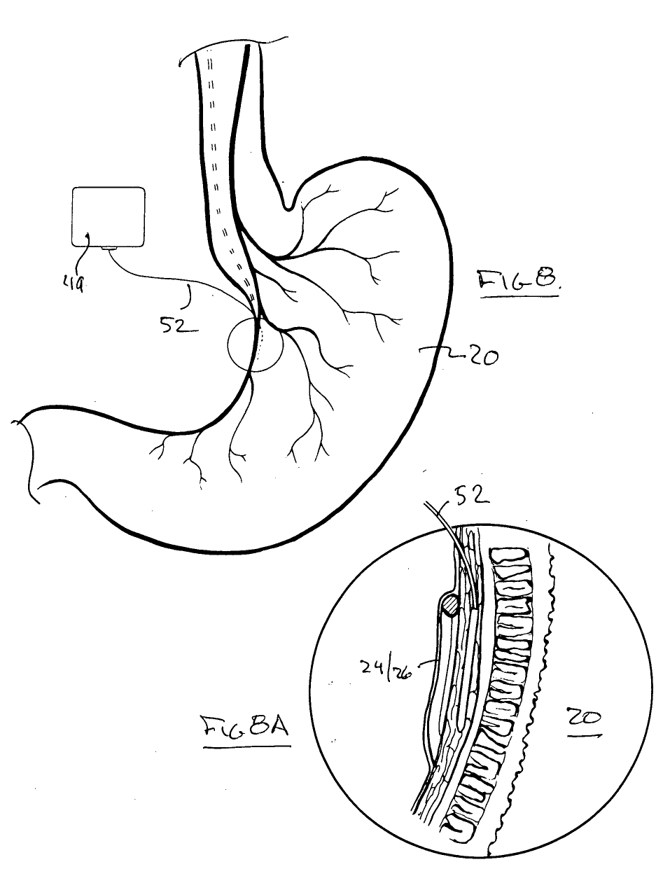

The piezoelectric effect, which here is a mechanical deformation of the bulking biomaterial creates electromechanical energy to both stimulate and “pace” the vagus nerve trunks 24/26 as would an implanted electrical pacemaker 49 as represented in FIG. 8, showing a sectional view representing an electrical pulse generator implanted in the abdominal wall. FIG. 8a shows this as an enlarged sectional view of an implanted electrical pacemaker electrode 52 shown in FIG. 8, adjacent the vagal nerve 24/26.

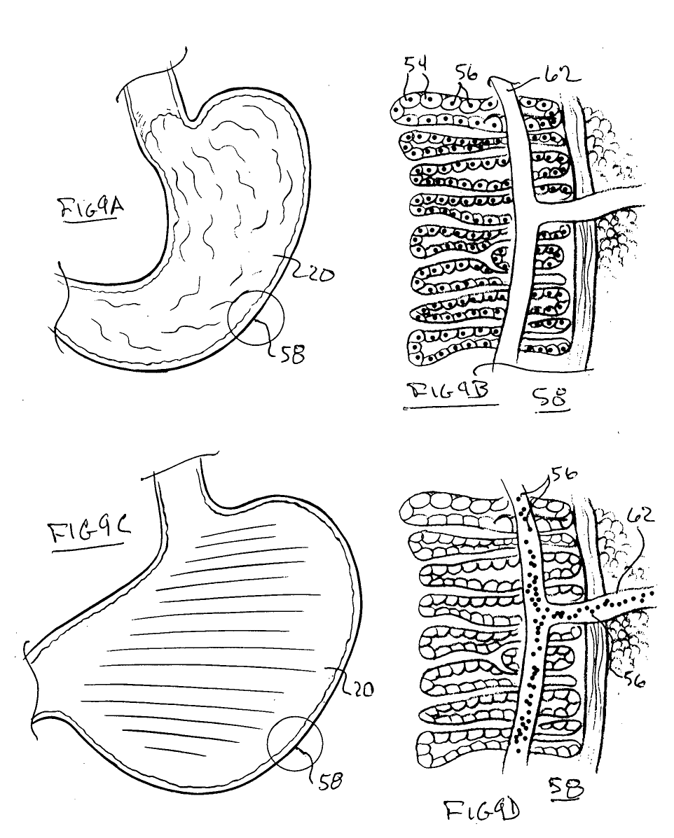

The FIG. 9a shows a sectional view of a preprandial (before meal) stomach 20, with FIG. 9B representing a sectional representation of ghrelin cells 54 and ghrelin 56 in the gastric layer 58 of the stomach 20 represented in FIG. 9A. FIG. 9C shows a sectional view of an enlarged “hungry” stomach 20 which is empty, and FIG. 9D represents a sectional view of empty “spent” ghrelin cells 60 and a ghrelin hormone 56 as it is released into the bloodstream 62 to create hunger and appetite at the hypothalmus in the person's brain.

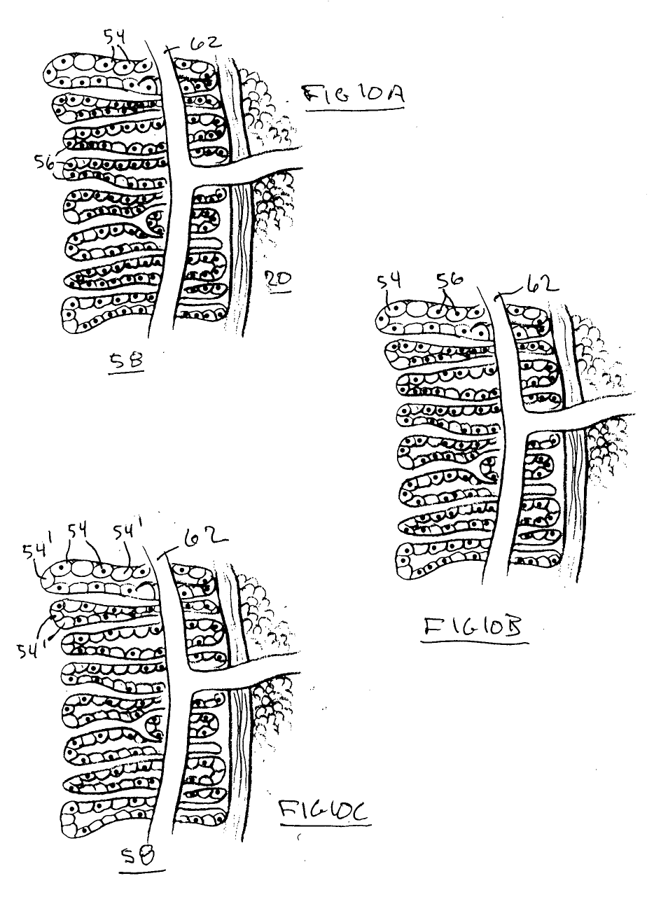

FIG. 10A is a sectional representation of the ghrelin cells 54 in the gastric layer 58 of a stomach 20, depicting about 90% of those cells 54 being capable of producing ghrelin 56 (before “bulking” of the vagal nerve trunk 24/26). FIG. 10b shows a sectional representation of the ghrelin cells 54 in the gastric layer 58 of a stomach 20, depicting only about 70% of the ghrelin cells 54 capable of producing ghrelin 56, about 30% of the ghrelin cells 54 becoming atrophied at a time of about one month after the vagal nerve bulking process, because of a decreased drawing power of ghrelin 56 through the vagal trunk 24/26. FIG. 10C shows a sectional representation of the ghrelin cells 54 in the gastric layer 58 of a stomach 20, depicting only about 40% of the ghrelin cells 54 capable of producing ghrelin 56, showing about 60% of the ghrelin cells 54′ atrophied and inactive after about three months time as a result of the inventive vagal nerve trunk bulking procedure of the present invention.

We claim:

1. A method for the appetite suppression of an individual comprising the steps of:
introducing a bulking material between the vagal nerve area and the stomach wall of a patient undergoing appetite suppression treatment to reduce production levels of ghrelin.

2. The method as recited in claim 1, wherein said vagal nerve area comprises the vagal trunk and the glandular layer of the stomach.

3. The method as recited in claim 1, wherein said vagal nerve area also comprises the vagal nerve arrayed in the lesser curvature of the stomach.

4. The method as recited in claim 1, including:
introducing a gastroscope through the gastroesophageal junction of a patient so as to inject a bulking agent adjacent the vagal nerve trunk to displace and compress said vagal nerve to reduce ghrelin levels in the patient's bloodstream.

5. A method for the appetite suppression of a patient, comprising:
displacing the vagal nerve trunk of the patient to effect a reduction in ghrelin levels in the system of the patient.

6. A method for the appetite suppression of a patient comprising:
compressing the vagal nerve trunk of a patient to effect a reduction in ghrelin levels in the system of the patient.
